(19) United States
(12) Patent Application Publication
(10) Pub. No.: US 2015/0182442 A1
(43) Pub. Date: Jul. 2, 2015

(54) TRADITIONAL CHINESE MEDICINE COMPOSITION THAT HAS THE EFFECT OF SOOTHING AND CLEANING FACE

(71) Applicant: Yuan Yuan, Beijing (CN)

(72) Inventor: Yuan Yuan, Beijing (CN)

(73) Assignee: Shang Chen Mei Bang (Beijing) Biotechnology Co., Ltd., Beijing (CN)

(21) Appl. No.: 14/144,556

(22) Filed: Dec. 31, 2013

(51) Int. Cl. A61K 8/97; A61Q 19/10; A61K 8/60; A61K 8/64; A61K 8/362

(52) U.S. Cl. CPC A61K 8/97; A61K 8/64; A61K 8/362; A61K 8/602; A61Q 19/10; A61K 2800/5922

(57) ABSTRACT

The invention provides a traditional Chinese medicine composition that has the effect of soothing and cleaning face, wherein said composition comprises the following servings by weight of the composition ratio of raw material: 5 to 15 servings of water, 2 to 4 servings of decyl glucoside, 1 to 2 servings of lotus extract, 3 to 5 servings of rose oil, 2 to 3 servings of calendula extract, 1 to 2 servings of Witch Hazel extract, 4 to 5 servings of glycerin, 3 to 5 servings of wheat germ extract, 2 to 3 servings of glycoproteins, 1 to 3 servings of citric acid, 2 to 3 servings peony root extract. The compositions can wash the face makeup and impurities, the skin to bring fresh and comfortable feeling, soft, smooth skin feel more compact, soft and moist.

### RELATED APPLICATIONS

This application claims the priority benefit of Chinese Patent Application Serial No. 201310703312.4, filed on Dec. 19, 2013; Chinese Patent Application Serial No. 201310685138.5, filed on Dec. 13, 2013; Chinese Patent Application Serial No. 201310687223.5, filed on Dec. 13, 2013; Chinese Patent Application Serial No. 201310685115.4, filed on Dec. 13, 2013; Chinese Patent Application Serial No. 201310684837.8, filed on Dec. 13, 2013; Chinese Patent Application Serial No. 201310685112.0, filed on Dec. 13, 2013; Chinese Patent Application Serial No. 201310686861.5, filed on Dec. 13, 2013; Chinese Patent Application Serial No. 201310687218.4, filed on Dec. 13, 2013; Chinese Patent Application Serial No. 201310686865.3, filed on Dec. 13, 2013; Chinese Patent Application Serial No. 201310687250.2, filed on Dec. 13, 2013; Chinese Patent Application Serial No. 201310684846.7, filed on Dec. 13, 2013; Chinese Patent Application Serial No. 201310685166.7, filed on Dec. 13, 2013; and Chinese Patent Application Serial No. 201310687246.6, filed on Dec. 16, 2013, the disclosure of all of which are hereby incorporated by reference in their entirety.

### FIELD OF THE INVENTION

The invention belongs to the field of traditional Chinese medicine, in particular to a traditional Chinese medicine composition that has the effect of soothing and cleaning face.

### BACKGROUND OF THE INVENTION

At present, our management and technical whitening cosmetics there is a big flaw, very serious violations added, some even to the amino mercuric chloride and other contraband as whitening agents such as hydrogen peroxide or peroxide bleach added over the years resulting in a Many beauty accidents, even disfigured, to consumers caused great harm, and has attracted national attention of consumers. In recent years, the whitening agent used in cosmetics mainly compound hydroquinone (hydroquinone), L-ascorbic acid (vitamin C), kojic acid, superoxide dismutase (SOD) and arbutin. However, there are many of these defects whitening agents, such as hydroquinone irritant very large, very unstable vitamin C and SOD, kojic acid also has some irritating and easy discoloration, arbutin role of single and poor effects. Therefore, the development has good security, stability, suppression effect is obvious, a good cost-effective natural whitening agent, has become one of the main research directions pharmaceutical and cosmetic industries, has a very good development prospects. Natural substances used in cosmetic skin treatments and skin care can achieve the double effect, but also versatile, widely applicable, and security features, thus increasing attention.

CN102225039A invention patent published licorice, moutan, Tribulus terrestris, Huang Jing containing motherwort whitening medicine composition for the Chinese medicine composition was prepared using boiling extraction method. Published patent CN101612105 skin whitening medicine composition and its preparation method of Baiji, grabbing, Morus alba, Poria, Tribulus terrestris, Atractylodes, white cardamom, white peony root, angelica Pyrolae and extract composition The compositions were prepared by the method of extraction and separation and enrichment resin soaked.

CN101732223A invention discloses a patent from the aloe vera, white lilac, white cows, Poria, white nuts, Baiji, Angelica, Atractylodes, bird's nest, barley, gentian, angelica, ginkgo composed of pure herbal whitening lotion, the medicine The composition was prepared using the boiling method of extraction.

CN101804185A invention discloses a patent containing Radix, angelica, white peony root, dogwood, Atractylodes, Poria, Chuanxiong, peach, white silkworm, simmer ginger, mint and licorice freckle medicine composition for the use of the use of Chinese medicine composition supercritical carbon dioxide extraction technology to extract.

### SUMMARY OF THE INVENTION

The present invention can wash away the face makeup and impurities, the skin to bring fresh and comfortable feeling, soft, smooth skin feel more compact, soft and moist. Techniques of the present invention are: the kind of traditional Chinese medicine composition has a soothing cleansing effect, wherein the active ingredient composition made according to the following servings by weight: 10-15 servings water, decyl glucoside 2-4 servings Lotus extract 1-2 servings, 3-5 servings rose oil, calendula extract 2-3 servings, 1-2 servings Witch Hazel Extract, Glycerin 4-5 servings, wheat germ extract 3-5 copies of glycoproteins 2-3 servings, 1-3 servings of citric acid, peony root extract 2-3 servings.

Wherein each component can be: 8-10 servings water, 2-3 servings of decyl glucoside, 1-2 servings lotus extract, rose oil 3-4, 2-3 servings of marigold extract, North Golden 1-2 servings strand plum extract, glycerin 4-5 servings, wheat germ extract 3-4, 2-3 servings glycoprotein, citric 1-2 copies, peony root extract 2-3 servings.

Wherein each component may be: 11-15 servings water, decyl glucoside 3-4, 1-2 servings lotus extract, 4-5 servings of rose oil, 2-3 servings of marigold extract, North Golden 1-2 servings strand plum extract, glycerin 4-5 servings, 4-5 servings of wheat germ extract, glycoproteins 2-3 servings, 2-3 servings of citric acid, peony root extract 2-3 servings.

The components are extracted through conventional methods together with accessories made Cleanser.

Also provides a composition for use of the medicine, sooner or later, apply to face and neck with warm water to fully wet hands and face, gently taking in the face and neck extended three pearl-sized amount in the hands of a dense foam after rubbing let cleanser and makeup and dirt full integration, then rinse with water.

Pharmacological Effects:

Decyl glucoside: excellent decontamination, foam, foam stabilization, emulsification, dispersion, solubilization, wetting, penetration ability. Acid. Alkali. Not sensitive to electrolytes. Reduce other irritating surfactants. No cloud point. Green surfactants. Alternatively AEO, AES, LAS and other surfactants. Intermediates in organic synthesis. And good skin compatibility.

Lotus extract: Qingre, bleeding, cure dysentery, reduce anger, in addition to alpine, supplement, stomach.

Rose oil is distilled extract of rose petals, high economic value. Can be used for medical, cosmetic, food, aromatherapy, research and other fields.

Calendula extract, calendula extract Compositae Marigold petals, has anti-aging effect.

Glycerol, glycerol i.e., the cytoskeletal component is a triglyceride molecule. When the edible fat intake body, wherein the body through the catabolism of triglyceride, glycerin is formed and stored in fat cells.

Glycoprotein: glycoprotein (glycoprotein) is a complex oligosaccharide chains of the polypeptide chain is covalently attached to the branched configuration, the main chain is shorter, in most cases, the sugar content is less than proteins.

Citric acid: It is an important organic acid, also known as citric acid, colorless crystals, often containing one molecule of water of crystallization, odorless, has a strong acidity, soluble in water. Calcium salt easily soluble in hot water than in cold water, the nature of the citric acid used to identify and isolate. Suitable crystallization temperature control obtained anhydrous citric acid. In industry, food industry, cosmetic industry, so has very many uses.

After the composition of the present invention, the above ingredients, a synergistic effect of its efficacy.

### DETAILED DESCRIPTION OF EMBODIMENT

Example 1: A medicine having a soothing effect cleansing composition, wherein the active ingredient is made according to the following servings of the composition by weight: 8-10 servings of water, 2-3 servings of decyl glucoside, lotus extract 1-2 servings, rose oil 3-4, 2-3 servings calendula extract, 1-2 servings Witch Hazel extract, glycerin 4-5 servings, 3-4 servings of wheat germ extract, sugar 2-3 servings protein, citric 1-2 copies, peony root extract 2-3 servings.

Example 2: The effect of a medicine having a soothing cleansing composition, wherein the active ingredient is made according to the following servings of the composition by weight: 11-15 servings of water, 3-4 servings of decyl glucoside, lotus extract 1-2servings, 4-5 servings of rose oil, calendula extract 2-3 servings, 1-2 servings of North America, witch hazel extract, glycerin 4-5 servings, 4-5 servings of wheat germ extract, sugar 2-3 servings protein, citric 2-3 servings, peony root extract 2-3 servings.

Usage: apply to face and neck morning and night with warm water to fully wet hands and face, take three pearl -sized amount in the hands gently rubbing the face and neck extended in the dense foam, so cleanser and makeup and dirt full integration, then rinse with water.

The kinds of Chinese medicine composition has a soothing cleansing effect after three years of clinical tests, recording 200 cases, in order to significantly improve the skin condition as a standard, the total efficiency of 98%. General medicine 7-15 days, can significantly improve the skin condition, skin to bring fresh and comfortable feeling, soft, smooth skin feel more compact, soft and moist.

The following referenced cases are herein incorporated: U.S. Nonprovisional patent application Ser. No. 14/xxx,xxx, filed Dec. 30, 2013 with Attorney Docket No. ShangChen-13-02, and entitled “A Traditional Chinese medicine extract composition with whitening skin care function and its preparation method”; U.S. Nonprovisional patent application Ser. No. 14/xxx,xxx, filed Dec. 30, 2013 with Attorney Docket No. Shangchen-13-03, and entitled “A traditional Chinese medicine composition for pigment repair”; U.S. Nonprovisional patent application Ser. No. 14/xxx,xxx, filed Dec. 30, 2013 with Attorney Docket No. Shangchen-13-04, and entitled “A traditional Chinese medicine composition for repair of acne”; U.S. Nonprovisional patent application Ser. No. 14/xxx,xxx, filed Dec. 30, 2013 with Attorney Docket No. Shangchen-13-05, and entitled “A traditional Chinese medicine composition for anti-wrinkle”; U.S. Nonprovisional patent application Ser. No. 14/xxx,xxx, filed Dec. 30, 2013 with Attorney Docket No. Shangchen-13-06, and entitled “A traditional Chinese medicine composition for dispelling wrinkles and black eye and its preparation method”; U.S. Nonprovisional patent application Ser. No. 14/xxx,xxx, filed Dec. 30, 2013 with Attorney Docket No. Shangchen-13-07, and entitled “A Chinese medicine composition for nourishing body care and its preparation method”; U.S. Nonprovisional patent application Ser. No. 14/xxx,xxx, filed Dec. 30, 2013 with Attorney Docket No. Shangchen-13-08, and entitled “Chinese medicine composition that having acne cleansing effect”; U.S. Nonprovisional patent application Ser. No. 14/xxx,xxx, filed Dec. 30, 2013 with Attorney Docket No. Shangchen-13-09, and entitled “An anti-aging skin rejuvenation medicine composition and its preparation method”; U.S. Nonprovisional patent application Ser. No. 14/xxx,xxx, filed Dec. 30, 2013 with Attorney Docket No. Shangchen-13-10, and entitled “Chinese medicine composition for pigment desalination”; U.S. Nonprovisional patent application Ser. No. 14/xxx,xxx, filed Dec. 30, 2013 with Attorney Docket No. Shangchen-13-011, and entitled “Chinese medicine composition for acne scar repair”; U.S. Nonprovisional Patent Application No. 14/xxx,xxx, filed Dec. 30, 2013 with Attorney Docket No. Shangchen-13-012, and entitled “Chinese medicine composition for Prevent from aging and soothing the skin”; and U.S. Nonprovisional patent application Ser. No. 14/xxx,xxx, filed Dec. 30, 2013 with Attorney Docket No. Shangchen-13-013, and entitled “Chinese medicine composition for Nourishing the skin and its preparation method”, the disclosure of all of which are herein incorporated by reference in its entirety.

What is claimed is:

1. A traditional Chinese medicine composition that has the effect of soothing and cleaning face, characterized in that the active ingredient composition made according to the following servings by weight: 10-15 servings water, decyl glucoside 2-4 servings, lotus extract 1-2 servings, rose oil 3-5 servings of marigold extract servings 2-3, 1-2 servings of hamamelis extract, glycerin 4-5 servings, 3-5 servings of wheat germ extract, glycoproteins 2-3 servings, 1-3 servings of citric acid, and peony root extract 2-3 servings.

2. Chinese medicine composition as claimed in claim 1, wherein the components in the following servings by weight: 8-10 servings of water, 2-3 servings of decyl glucoside, 1-2 servings lotus extract, rose oil 3-4 servings, 2-3 servings of marigold extract, hamamelis extract servings 1-2, 4-5 servings of glycerin, wheat germ extract 3-4, 2-3 servings of glycoproteins, citric acid 1-2 copies, and peony root extract 2-3 servings.

3. Chinese medicine composition as claimed in claim 1, wherein the ingredients in the following servings by weight: 11-15 servings of water, decyl glucoside, 3-4, 1-2 servings lotus extract, rose oil servings 4-5, 2-3 servings of marigold extract, hamamelis extract servings 1-2, 4-5 servings glycerin 4-5 servings of wheat germ extract, glycoproteins 2-3 servings citrate 2-3 servings, peony root extract 2-3 servings.

4. The medicine composition according to claim 1, wherein the components through a conventional method together with auxiliary materials cleanser.

5. The Chinese medicine composition according to claim 1, which is applied to the face and neck morning and night with warm water to fully wet hands and face, take three pearl-sized amount in the hands knead dense foam After gently extended in the face and neck, let cleanser and makeup and dirt full integration, then rinse with water.
